(19) United States
(12) Patent Application Publication
(10) Pub. No.: US 2025/0288440 A1
(43) Pub. Date: Sep. 18, 2025

(54) Method for manufacturing stent delivery system and stent delivery system

(71) Applicant: TERUMO KABUSHIKI KAISHA, Tokyo (JP)

(72) Inventors: Takuya KUROKI, Fujinomiya-shi (JP); Erena SHIMOJI, Newton, MA (US); Ryota IKEUCHI, Newton, MA (US); Yoichiro KUWANO, Princeton, NJ (US)

(73) Assignee: TERUMO KABUSHIKI KAISHA, Tokyo (JP)

(21) Appl. No.: 19/075,896

(22) Filed: Mar. 11, 2025

(30) Foreign Application Priority Data
Mar. 13, 2024 (JP) 2024-039224

(51) Int. Cl. A61F 2/95; A61F 2/958

(52) U.S. Cl. CPC A61F 2/9522; A61F 2/958

(57) ABSTRACT

A method for manufacturing a stent delivery system is a method for manufacturing a stent delivery system that includes a balloon catheter including a balloon and a shaft supporting the balloon, and a stent disposed on the balloon and formed in a tubular shape, the balloon being joined to the shaft at an end portion in an extending direction of the shaft. The method includes a first diameter reduction step of compressing the stent inward in a radial direction of the stent to reduce diameters of the stent and the balloon in a state where the balloon is inserted into a tube of the stent, and then releasing the compression. In the first diameter reduction step, the stent is compressed to a first diameter, the first diameter being equal to or smaller than a diameter of a proximal-side joint portion where the balloon is fused to the shaft.

### CROSS-REFERENCES TO RELATED APPLICATIONS

This application claims priority to Japanese Application No. 2024-039224 filed on Mar. 13, 2024, the entire content of which is incorporated herein by reference.

### TECHNOLOGICAL FIELD

The present disclosure relates to a method for manufacturing a stent delivery system and a stent delivery system.

### BACKGROUND DISCUSSION

Japanese Patent Application Publication No. 2012-070912 A discloses a method for manufacturing a stent delivery system in which a stent is crimped on a balloon catheter. In the method for manufacturing a stent delivery system, the prepared stent is deflated in advance to a second diameter having an inner diameter equal to or smaller than an outer diameter of a folded balloon, the folded balloon is inserted into the stent, and pressure is further applied radially inward from an outer surface of the stent to deflate the stent to a third diameter which is a diameter at which crimping is completed. It is considered that it is possible to manufacture a stent delivery system capable of preventing the stent from falling off or moving in this method for manufacturing a stent delivery system.

In the stent delivery system, when the diameter of the stent in the state of being crimped on the balloon of the balloon catheter is large, the performance of the balloon catheter in passing through a lesion of the balloon catheter deteriorates in some cases. However, a decrease in diameter is not sufficient in the related art as disclosed in Japanese Patent Application Publication No. 2012-070912 A, for example. Therefore, it is desired to decrease the diameter of the balloon catheter on which the stent is crimped.

### SUMMARY

A method for manufacturing a stent delivery system and a stent delivery system which achieve a decrease in diameter of a balloon catheter on which a stent is crimped.

The method for manufacturing a stent delivery system and the stent delivery system according to the present disclosure for achieving the above object are as follows.

[1] A method for manufacturing a stent delivery system including a balloon catheter that includes a balloon inflated or deflated by supply or discharge of fluid and a shaft that supports the balloon and supplies or discharges the fluid, and a stent disposed on the balloon and formed in a tubular shape, the balloon being joined to the shaft at an end in an extending direction of the shaft, the method including a first diameter reduction step of compressing the stent inward in a radial direction of the stent in a state where the balloon is inserted into a tube of the stent to reduce diameters of the stent and the balloon, and then releasing the compression, wherein the stent is compressed to a first diameter in the first diameter reduction step, and the first diameter is equal to or smaller than a diameter of a joint portion where a proximal portion of the balloon is joined to the shaft.

[2] The method for manufacturing a stent delivery system according to [1], further including a second diameter reduction step performed after the first diameter reduction step, the second diameter reduction step of compressing the stent inward in the radial direction of the stent to reduce the diameters of the stent and the balloon, and then releasing the compression, wherein the stent is compressed to a second diameter in the second diameter reduction step, and the second diameter is equal to or smaller than the diameter of the joint portion.

[3] The method for manufacturing a stent delivery system according to [2], wherein the second diameter reduction step is repeated twice (two times) or more.

[4] The method for manufacturing a stent delivery system according to [2] or [3], wherein a start diameter at the compression in the second diameter reduction step is equal to or smaller than the diameter of the joint portion.

[5] The method for manufacturing a stent delivery system according to any one of [2] to [4], wherein when a change rate of the diameter of the stent when a load of 5 N or more and 10 N or less per length of 1 mm in an axial direction of the stent is applied inward in the radial direction of the stent is 0.60% or less, repetition of the second diameter reduction step is ended.

[6] A stent delivery system including: a balloon catheter that includes a balloon inflated or deflated by supply or discharge of fluid and a shaft that supports the balloon and supplies or discharges the fluid; and a stent disposed on the balloon and formed in a tubular shape, wherein an end of the balloon in an extending direction of the shaft is joined to the shaft, and a diameter of the stent is equal to or smaller than a diameter of a joint portion where a proximal portion of the balloon is joined to the shaft.

[7] A method for manufacturing a stent delivery system including a balloon catheter that includes a balloon inflated or deflated by supply or discharge of fluid and a shaft that supports the balloon and supplies or discharges the fluid, and a stent disposed on the balloon and formed in a tubular shape, the balloon being joined to the shaft at an end in an extending direction of the shaft, the method comprising: compressing the stent inward a first time in a radial direction of the stent in a state where the balloon is inserted into a tube of the stent to reduce diameters of the stent and the balloon, and then releasing the compression.

According to the present disclosure, it is possible to provide the method for manufacturing a stent delivery system and the stent delivery system that achieve the decrease in diameter of the balloon catheter on which the stent is crimped.

### BRIEF DESCRIPTION OF THE DRAWINGS

FIG. 1 is an explanatory view of a stent delivery system.

FIG. 2 is a cross-sectional view taken along line II-II in FIG. 1.

FIG. 3 is a cross-sectional view of a balloon, a stent, a portion of a shaft supporting the balloon, and the periphery of these members.

FIG. 4 is an exploded view of the stent.

FIG. 5 is an explanatory view of states of the balloon, the stent, and a crimping head before a diameter of the stent is reduced in a crimping operation.

FIG. 6 is an explanatory view of states of the balloon, the stent, and the crimping head after the diameter of the stent is reduced in the crimping operation.

FIG. 7 is a graph illustrating a method for manufacturing the stent delivery system in the relationship between an opening diameter of the crimping head and step elapsed time.

FIG. 8 is a cross-sectional view of another stent delivery system.

### DETAILED DESCRIPTION

A method for manufacturing a stent delivery system according to an embodiment of the present disclosure will be described with reference to the drawings.

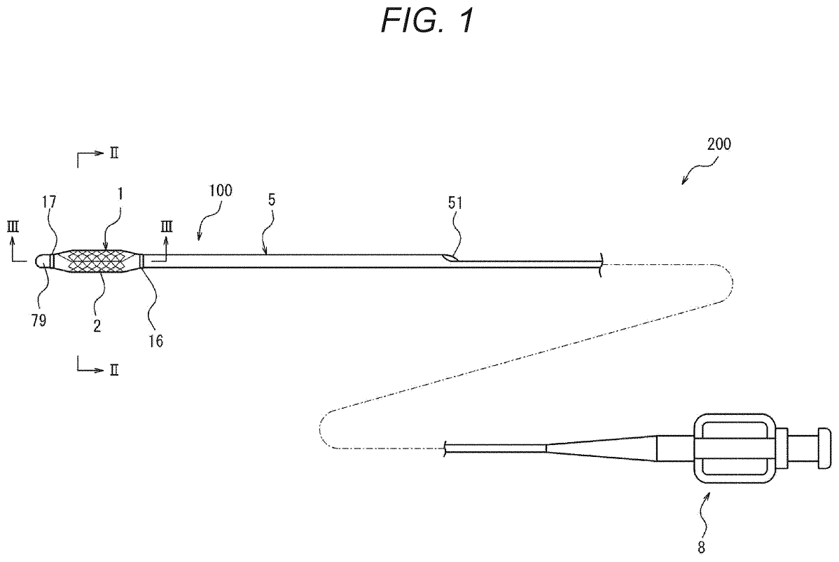

FIG. 1 illustrates a stent delivery system 200 achieved by the method for manufacturing a stent delivery system according to the present embodiment.

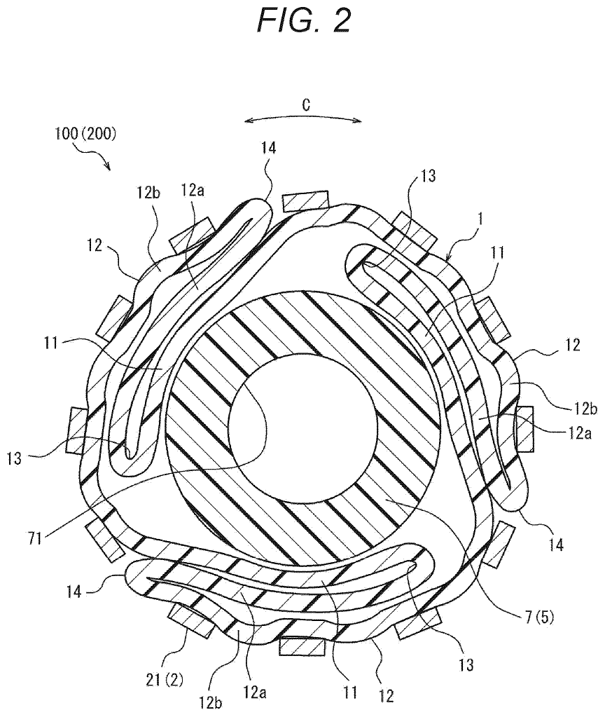

FIG. 2 is a cross-sectional view taken along line II-II in FIG. 1. First, an outline of the stent delivery system 200 and the method for manufacturing the same will be described.

As illustrated in FIG. 1, the stent delivery system 200 includes a balloon catheter 100 having a balloon 1 inflated or deflated by supply or discharge of fluid, and a stent 2 disposed on the balloon 1 and formed in a tubular shape.

As illustrated in FIG. 2, the stent 2 allows the balloon 1 inserted into a tube (i.e., tubular portion) of the stent 2, and is fixed on an outer peripheral surface of the balloon 1. That is, the stent 2 is crimped on the balloon 1. Note that the balloon 1 is in a deflated state in FIGS. 1 and 2.

The stent delivery system 200 can be manufactured by the method for manufacturing a stent delivery system, the method including a diameter reduction step of compressing the stent 2 inward in a radial direction of the stent 2 to reduce diameters of the stent 2 and the balloon 1 and then releasing the compression in a state where the balloon 1 is inserted into the tube of the stent 2 formed in the tubular shape.

Hereinafter, the stent delivery system and the method for manufacturing the same will be described in detail.

The stent delivery system 200 including the balloon catheter 100 illustrated in FIG. 1 is a medical device used for a procedure (for example, PCI (percutaneous coronary intervention)) of pushing and widening a lesion (stenosed site) formed in a living body lumen such as a blood vessel. In the procedure using the stent delivery system 200, an operator inserts the stent 2 crimped on the balloon 1 disposed at a distal portion of a shaft 5 into the living body lumen. The operator expands the balloon 1 on the inner peripheral side of the stenosed site formed in the living body lumen, and expands the stent 2 together with the balloon 1. The operator can maintain the stenosed site being pushed and widened by placing the expanded stent 2 on the inner peripheral side of the stenosed site.

Note that the balloon catheter 100 is used to deliver the stent 2 to the stenosed site, but can also be configured to be used for the purpose of, for example, treating and improving a stenosed site formed in a living body organ such as a blood vessel, a bile duct, a trachea, an esophagus, other digestive tract, a urethra, a lumen in the ears and nose, or other organs.

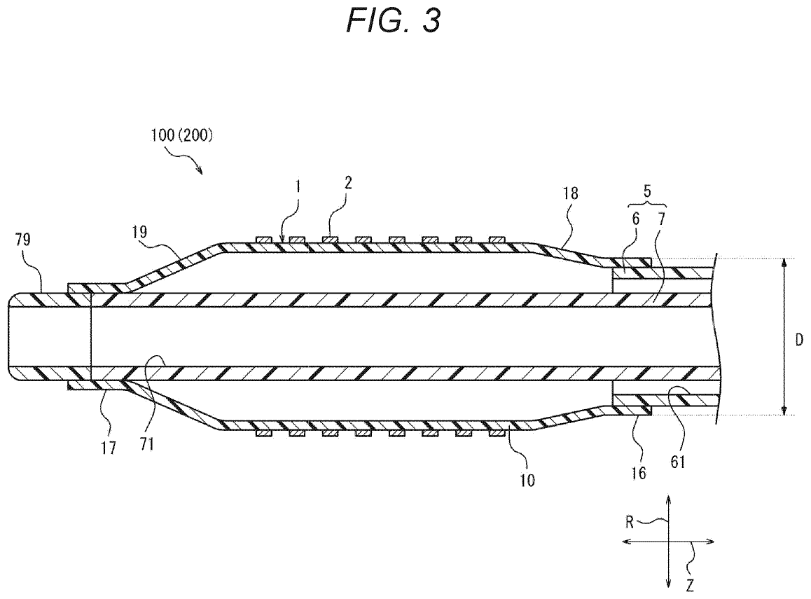

As illustrated in FIGS. 1 to 3, the balloon catheter 100 includes the shaft 5 having flexibility and an elongated shape, the balloon 1 disposed at the distal portion of the shaft 5, and a hub 8 (see FIG. 1) disposed at a proximal portion of the shaft 5. In the stent delivery system 200 (see FIG. 1), the stent 2 is crimped on the balloon 1 of the balloon catheter 100. Note that FIG. 3 is a cross-sectional view at a position corresponding to arrow III-III in FIG. 1, and is a cross-sectional view of the balloon 1 in an expanded state, the stent 2, and a portion of the shaft 5 supporting the balloon 1 and the periphery of these members. FIG. 3 illustrates a cross section overlapping an axial center of the shaft 5.

The balloon catheter 100 may be provided with a guide wire port 51 through which a guide wire or the like is led out closer to the distal portion side of the shaft 5.

As illustrated in FIG. 3, the shaft 5 includes an inner tube 7 in which a lumen 71 through which the guide wire or the like is inserted is formed, and an outer tube 6 forming a lumen 61 through which a pressurizing medium (for example, fluid such as saline or contrast) can flow between the inner tube 7 and the outer tube 6. The shaft 5 has a double tube structure in which the inner tube 7 and the outer tube 6 are concentrically disposed by inserting the inner tube 7 into the outer tube 6.

The shaft 5 supports the balloon 1. The inner tube 7 of the shaft 5 passes through the balloon 1. The shaft 5 supplies the above-described fluid to a space in the balloon 1 or discharges the fluid from the balloon 1, thereby inflating or deflating the balloon 1.

The balloon 1 is liquid-tightly and airtightly joined to a distal portion of the inner tube 7 by welding or the like. A distal portion of the balloon 1 in an extending direction of the shaft 5 is joined to the inner tube 7 by fusion or the like. In addition, a proximal portion of the balloon 1 in the extending direction of the shaft 5 is liquid-tightly and airtightly joined to the outer tube 6 by fusion or the like. In FIG. 3, a portion of the balloon 1 where the distal portion of the balloon 1 and the inner tube 7 are joined is illustrated as a distal-side joint portion 17. In addition, a portion of the balloon 1 where the proximal portion of the balloon 1 and the outer tube 6 are joined is illustrated as a proximal-side joint portion 16. A diameter D of the proximal-side joint portion 16 is larger than a diameter (outer diameter) of the distal-side joint portion 17.

A distal tip 79 may be attached to a distal end of the inner tube 7 to protect a biological organ (for example, an inner wall of a blood vessel) from damage when, for example, a distal end of the balloon catheter 100 comes into contact with the biological organ. The distal tip 79 can be configured using, for example, a resin material more flexible than that of the inner tube 7.

The pressurizing medium can flow into a space (hereinafter, referred to as an internal space) between the balloon 1 and the inner tube 7.

The balloon 1 is inserted into the living body lumen and is folded to keep the performance in passing through the inside of the living body lumen until reaching the stenosed site in the living body lumen.

The balloon 1 expands when the pressurizing medium flows into the internal space (see FIG. 3). When the balloon 1 expands, the balloon catheter 100 is expanded in diameter such that a part of the balloon 1 presses the stent 2 against the stenosed site formed in the living body lumen. The stent 2 is placed in a state of being expanded in diameter by the balloon 1 while pushing and widening the stenosed site.

The balloon 1 is inserted into the living body lumen and is folded in a deflated state in order to keep the performance in passing through the inside of the living body lumen until reaching the stenosed site in the living body lumen (see FIG. 2). Note that the deflated state of the balloon 1 is a state where the pressurizing medium does not flow into the internal space.

As illustrated in FIG. 2, the balloon 1 may be divided into three or more regions and folded along a circumferential direction (direction C in FIG. 2) of the inner tube 7. In FIG. 2, each of the regions of the balloon 1 is folded so as to have a wing base 11 adjacent to the inner tube 7 and along the inner tube 7, and a wing 12 stacked on the wing base 11.

The wing 12 in each of the regions is stacked on the wing base 11 along the same direction in the circumferential direction. The wing 12 has an inner portion 12a is stacked adjacently on the wing base 11 and an outer portion 12b stacked on the inner portion 12a.

A boundary portion between the wing base 11 and the wing 12, that is, the boundary portion between the wing base 11 and the wing 12 placed on the wing base 11 is a fold portion 13 along an axial direction (direction Z in FIG. 3) of the inner tube 7. The fold portion 13 is located on the inner side of the outer portion 12b in a radial direction (direction R in FIG. 3) of the inner tube 7.

An end portion of the inner portion 12a, which is a boundary portion between the inner portion 12a and the outer portion 12b and on the opposite side of the fold portion 13 in the circumferential direction of the inner tube 7, is a fold portion 14 along the axial direction of the inner tube 7.

As illustrated in FIG. 3, as for a size of the balloon 1 in the time of expansion, for example, a straight portion 10 has an outer diameter of about 1 mm to 20 mm and preferably about 1 mm to 10 mm, and a length in the axial direction (direction Z) of about 5 mm to 100 mm and preferably about 5 mm to 60 mm. In addition, the distal-side joint portion 17 has an outer diameter of about 0.3 mm to 1.5 mm and preferably about 0.5 mm to 1.3 mm, and a length in the axial direction of about 0.5 mm to 5 mm and preferably about 0.5 mm to 3 mm. The proximal-side joint portion 16 has an outer diameter of about 0.5 mm to 1.8 mm and preferably about 0.6 mm to 1.3 mm, and a length in the axial direction of about 1 mm to 8 mm and preferably about 1 mm to 6 mm. Furthermore, lengths of a distal tapered portion 19 and a proximal tapered portion 18 are about 1 mm to 10 mm and preferably about 3 mm to 7 mm.

As a material for forming the balloon 1, for example, an organic polymer material can be used. As the organic polymer material forming the balloon 1, specifically, a polymer material such as polyolefins (for example, polyethylene, polypropylene, polybutene, an ethylene-propylene copolymer, an ethylene-vinyl acetate copolymer, an ionomer, or a mixture of two or more kinds of polyolefins), polyvinyl chloride, polyamide, polyamide elastomer, polyurethane, polyurethane elastomer, polyimide, or fluororesin, or a mixture of the polymer materials described above, or an elastic resin material such as the two or more polymer materials described above can be used, and among them, a polyamide-based resin can be suitably used as a main material.

The stent 2 is a member formed in the tubular shape as illustrated in FIG. 3. The stent 2 is made of a metal alloy as an example. The stent 2 is a wire mesh member formed in a tubular shape as an example. The balloon 1 is inserted into the tube of the stent 2. The stent 2 is fixed (crimped) on an outer surface of the balloon 1. In a state where the stent 2 is fixed on the outer surface of the balloon 1, the radial direction of the stent 2 is the same as the radial direction of the inner tube 7. Hereinafter, the radial direction of the stent 2 and the radial direction of the inner tube 7 is sometimes collectively referred to simply as the radial direction.

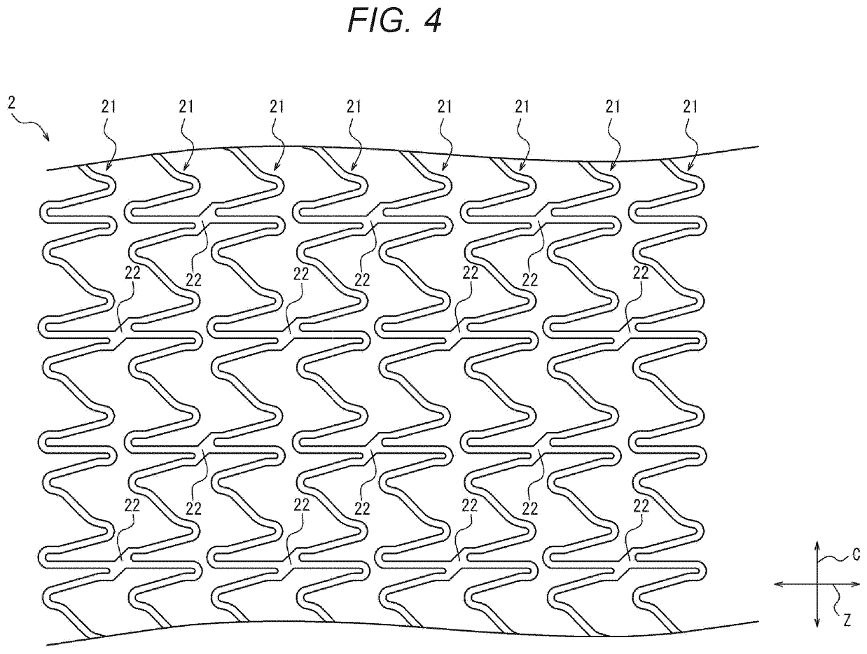

FIG. 4 illustrates an exploded view of the stent 2. As illustrated in FIG. 4, the stent 2 includes a plurality of annular portions 21 extending in a wave shape in the circumferential direction of the inner tube 7 (see FIG. 2) and disposed at predetermined intervals in the axial direction of the inner tube 7, and a plurality of link portions 22 each connecting the adjacent annular portions 21 to one another in the axial direction. The stent 2 is configured in the tubular shape. The balloon 1 is inserted inside the tube of the stent 2 (see FIGS. 2 and 3).

The stent 2 is designed to maintain the state of being fixed (crimped) on the outer surface of the balloon 1 and to maintain its expanded diameter once expanded by the balloon 1. Therefore, as the material of the stent 2, a material that is plastically deformed by expansion of the balloon 1 and maintains a shape after the expansion of the balloon 1 can be selected.

The metal alloy forming the stent 2 may contain at least one or more selected from cobalt, chromium, nickel, tungsten, molybdenum, iron, and platinum as an alloy component. Particularly suitable as the metal alloy forming the stent 2 can be, for example, L-605 alloy which is an alloy containing cobalt, chromium, tungsten and nickel.

The expanded diameter of the stent 2 is not particularly limited, and is, for example, 1 mm to 30 mm in outer diameter. The stent 2 according to the present embodiment is used to treat a lesion such as a stenosed site or an occluded site generated in a blood vessel, a bile duct, a trachea, an esophagus, a urethra, or other living body lumens. The expanded diameter of the stent 2 is set according to a diameter of the target lesion.

A diameter of the stent 2 in a state of being attached to the balloon catheter 100 before expansion is not particularly limited, and is, for example, 0.5 mm to 3 mm in outer diameter. When a diameter of a lumen in which the target lesion is present is small, the diameter in the state of being attached to the balloon catheter 100 also needs to be small.

A diameter of the stent 2 in a state before expansion and before being attached to the balloon catheter 100 is not particularly limited, and can be, for example, 1 mm to 5 mm in outer diameter. The stent 2 is manufactured using a known technique, and can be manufactured, for example, by removing an unnecessary portion other than a region to form a strut from a pipe material and then polishing the resultant. Thereafter, the stent 2 is reduced in diameter and attached to the balloon catheter 100. In this case, the outer diameter in the state before being attached to the balloon catheter 100 is an outer diameter of the stent 2 after being polished and is substantially the same as an outer diameter of the pipe material.

A thickness of the stent 2 is not particularly limited, and can be, for example, 0.05 mm to 1 mm.

A line width of the stent 2 is not particularly limited, and can be, for example, 0.05 mm to 1 mm.

An angle formed by a bent portion (hereinafter, referred to as a bent portion) in the annular portion 21 of the stent 2 is not particularly limited, and can be, for example, 10° to 120° at the time of uniform expansion.

An axial length of the link portion 22 is not particularly limited, and can be, for example, 0.05 mm to 50 mm. The link portion 22 may have a circumferentially extending component, but a circumferential length at that time is not particularly limited, and can be, for example, 0.05 mm to 50 mm.

A surface of the stent 2 may be coated with a drug such as an immunosuppressive agent. As a result, restenosis in the living body lumen after stent placement is prevented. Since the bent portion has a risk that stress concentration occurs at the time of expansion and the drug is peeled off, the bent portion is not necessarily coated with the drug.

In addition, for example, as in a stent graft, a cover member made of a fibrous material, a sheet-like material, or the like may be disposed on an outer surface or an inner surface of the stent 2. The permeability of the cover member to liquid or gas may be high or low. In addition, the cover member may be impermeable.

The stent 2 is crimped on the balloon 1 as follows. Hereinafter, as a method for manufacturing the stent delivery system 200, a method (hereinafter, referred to as a crimping method) of crimping the stent 2 by crimping the stent 2 to the balloon 1 will be described.

The crimping method according to the present embodiment includes a first diameter reduction step of compressing the stent 2 inward in the radial direction of the stent 2 to reduce the diameters of the stent 2 and the balloon 1 in the state where the balloon 1 is inserted into the tube of the stent 2, and then releasing the compression. In addition, the crimping method according to the present embodiment may include a second diameter reduction step performed after the first diameter reduction step to compress the stent 2 inward in the radial direction of the stent 2 to reduce the diameters of the stent 2 and the balloon 1, and then release the compression. The first diameter reduction step and the second diameter reduction step are operations of crimping the stent 2 to the balloon 1.

The first diameter reduction step is the operation including initial compression of the stent 2 and release of the compression. In the first diameter reduction step, the balloon 1 is provided with folds of the fold portions 13 and 14 (see FIG. 2) (the balloon 1 has a fold-up shape). The second diameter reduction step is an operation including compression and release of the compression performed after the first diameter reduction step. The second diameter reduction step may be repeated twice (two times) or more. The operation of reducing the diameters of the stent 2 and the balloon 1 in the first diameter reduction step and the operation of reducing the diameters of the stent 2 and the balloon 1 in the second diameter reduction step are the same operation in the basic part and are partially different.

The first diameter reduction step includes a first compression step of compressing the stent 2 inward in the radial direction of the stent 2 to reduce the diameter of the stent 2 to a first diameter, and a first release step of releasing the compression after the first compression step.

The first diameter reduction step may include a first maintenance step of maintaining the stent 2 at a first diameter. That is, the first diameter reduction step may include the first compression step of compressing the stent 2 inward in the radial direction of the stent 2 to reduce the diameter of the stent 2 to the first diameter, the first maintenance step of maintaining the stent 2 at the first diameter, and the first release step of releasing the compression after the first maintenance step. The first compression step, the first maintenance step, and the first release step are performed in this order.

In addition, the second diameter reduction step includes a second compression step of compressing the stent 2 inward in the radial direction of the stent 2 to reduce the diameter of the stent 2 to a second diameter, and a second release step of releasing the compression after the second compression step. When the second diameter reduction step is performed in addition to the first diameter reduction step, it is possible to further decrease the diameter of the stent 2 as the folds of the respective fold portions 13 and 14 (see FIG. 2) of the balloon 1 become settled or the space in the balloon 1 decreases, as compared with a case where only the first diameter reduction step is performed. In addition, through the second diameter reduction step, an amount of the balloon 1 pinched by the stent 2 increases or the like, so that the stent 2 is more firmly fixed to the balloon 1. That is, a holding force (retention) of the balloon catheter 100 for holding the stent 2 on the balloon 1 is improved.

Hereinafter, the improvement in the holding force for holding the stent 2 on the balloon 1 in the balloon catheter 100 is sometimes simply referred to as improvement in retention.

The second diameter reduction step is preferably repeated twice (two times) or more. As a result, the diameter is further decreased, or the retention is further improved. When the second diameter reduction step is performed three times (3) or more and twenty (20) times or less (i.e., 3 times to 20 times), a sufficient decrease in the diameter and sufficient improvement in the retention can be achieved.

The second diameter reduction step may include a second maintenance step of maintaining the stent 2 at the second diameter. That is, the second diameter reduction step may include the second compression step of compressing the stent 2 inward in the radial direction of the stent 2 to reduce the diameter of the stent 2 to the second diameter, the second maintenance step of maintaining the stent 2 at the second diameter, and the second release step of releasing the compression after the second maintenance step. The second compression step, the second maintenance step, and the second release step are performed in this order.

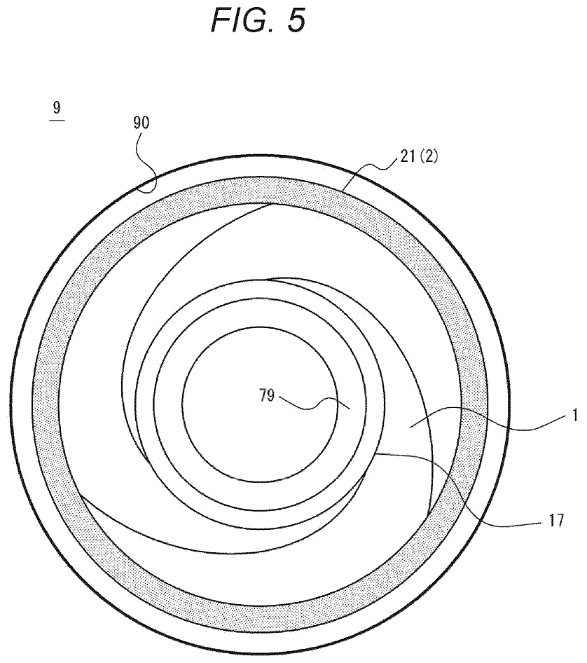

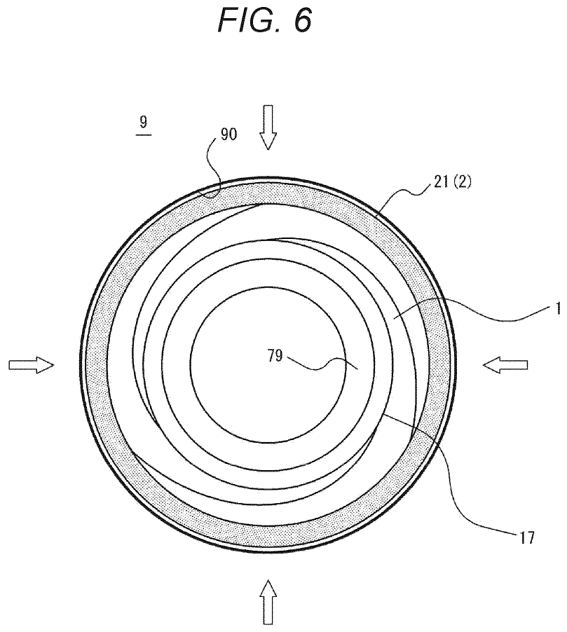

The diameter reduction of the stent 2 in the first diameter reduction step (first compression step) and the second diameter reduction step (first compression step) may be a crimping operation by a crimping head 9 of a crimping device as illustrated in FIGS. 5 and 6. Note that FIGS. 5 and 6 are explanatory views of images of states of the balloon 1, the stent 2, and the crimping head 9 before the diameter of the stent 2 is reduced and after the diameter of the stent 2 is reduced in this order in the crimping operation.

The crimping head 9 has a hole portion 90 having a straight body shape whose inner diameter can be reduced and enlarged. The diameter reduction of the stent 2 can be performed by inserting the balloon 1 to which the stent 2 is fixed into the hole portion 90, reducing a diameter (hereinafter, sometimes referred to as an opening diameter of the crimping head 9) of the hole portion 90, and pressing, that is, compressing an outer peripheral surface of the stent 2 along the radial direction. The compression of the stent 2 can be released by enlarging the opening diameter of the crimping head 9.

The diameter reduction of the stent 2 in each of the first diameter reduction step and the second diameter reduction step is preferably performed by applying a load of 5 N (newtons) or more and 10 N or less (5 N to 10 N) per length of 1 mm in the axial direction (direction Z in FIG. 3) of the stent 2 inward in the radial direction. As a result, the stent 2 can be crimped on the balloon 1 without damaging the balloon 1, and the diameters (outer diameters) of the stent 2 and the balloon 1 can be decreased. Hereinafter, decreasing the diameter of the stent 2 is sometimes simply referred to as a decrease in diameter or decreasing the diameter. The concept of the decrease in diameter or decreasing the diameter includes the case of decreasing the diameters of the stent 2 and the balloon 1. In the following description, when simply described as the diameter of the stent 2, it means the outer diameter of the stent 2.

In the first diameter reduction step, the opening diameter of the crimping head 9 may be reduced to the first diameter. As a result, the diameter reduction of the stent 2 can be performed until the diameter of the stent 2 reaches the first diameter.

The first maintenance step is a step of stopping, after the diameter of the stent 2 reaches the first diameter in the first compression step, the compression in this state and maintaining the diameter of the stent 2 at the first diameter for a certain period of time. As a result, the amount of the balloon 1 pinched by the stent 2 increases or the like, so that the stent 2 is more firmly fixed to the balloon 1. That is, the retention is improved. In the first maintenance step, a load of 5 N or more and 10 N or less (5 N to 10 N) per length of 1 mm in the axial direction of the stent 2 may be applied to the stent 2 inward in the radial direction.

In the second diameter reduction step, the opening diameter of the crimping head 9 may be reduced to the second diameter. That is, in the second diameter reduction step, the stent 2 may be compressed until the diameter of the stent 2 becomes the second diameter. As a result, the diameter reduction of the stent 2 can be performed until the diameter of the stent 2 reaches the second diameter.

The second maintenance step is a step of stopping, after the diameter of the stent 2 reaches the second diameter in the second compression step, the compression in this state and maintaining the diameter of the stent 2 at the second diameter for a certain period of time. This improves the retention. In the second maintenance step, a load of 5 N or more and 10 N or less (5 N to 10 N) may be applied to the stent 2 inward in the radial direction per length of 1 mm in the axial direction of the stent 2.

Incidentally, the second diameter may be equal to or smaller than the first diameter. That is, the second diameter may be the same as the first diameter. The second diameter may be smaller than the first diameter. The second diameter may be, for example, 3.0 mm or smaller. Hereinafter, a target value of the diameter reduction in each of the diameter reduction steps, such as the first diameter and the second diameter, is sometimes referred to as a final diameter.

The final diameter is preferably equal to or smaller than the diameter D which is an outer diameter of the proximal-side joint portion 16. The final diameter is preferably smaller than the diameter D of the proximal-side joint portion 16. As a result, the diameter can be decreased. For example, there is a case where it is possible to provide the stent delivery system 200 in which the diameter of the stent 2 is equal to or smaller than the diameter D of the proximal-side joint portion 16 and equal to or larger than the diameter of the inner tube 7. Note that it is preferable that the stent 2 and the outer tube 6 do not overlap in the radial direction when the final diameter is equal to or smaller than the diameter D of the proximal-side joint portion 16.

As described above, the second diameter reduction step may be repeated twice (two times) or more. In this case, the second diameter in a certain second diameter reduction step may be made smaller than the second diameter in the immediately previous second diameter reduction step. That is, the final diameter may be sequentially reduced as the second diameter reduction step is repeated.

The first release step and the second release step are steps of releasing the compression after compressing the stent 2 to the final diameter. Here, releasing the compression means ending the pressing to relax the stent 2 and the balloon 1, and is specifically performed by making the opening diameter of the crimping head 9 larger than the final diameter. When the opening diameter of the crimping head 9 is enlarged, the diameters of the stent 2 and the balloon 1 are enlarged (so-called recoil). Note that the recoil means that the stent 2 naturally expands in diameter by an amount of deformation due to elastic deformation at the time of diameter reduction of the stent 2.

After the first diameter reduction step (after the first release step), the diameters (diameters after the recoil) of the stent 2 and the balloon 1 are smaller than those at the start of the first diameter reduction step. Therefore, when the compression is released in the first diameter reduction step, that is, in the first release step, a diameter (hereinafter, referred to as a first release diameter) of the hole portion 90 at the end of the first release step may be smaller than a diameter (hereinafter, referred to as a start diameter) of the hole portion 90 at the start of the first diameter reduction step. Note that the start diameter is the opening diameter of the crimping head 9 at the time when an inner surface of the hole portion 90 comes into contact with the entire outer peripheral surface of the stent 2, in other words, at the time when the compression of the stent 2 is started.

As described above, after the first diameter reduction step, the diameters of the stent 2 and the balloon 1 (diameters after the recoil) are smaller than those at the start of the first diameter reduction step, and thus, the opening diameter of the crimping head 9 at the start of the second diameter reduction step (the diameter of the hole portion 90, hereinafter referred to as a second start diameter) is smaller than the start diameter. In the present embodiment, the second start diameter of the first second diameter reduction step is the same as the first release diameter.

The second start diameter is preferably equal to or smaller than the diameter D of the proximal-side joint portion 16. The second start diameter is preferably smaller than the diameter D of the proximal-side joint portion 16. As a result, the diameter can be decreased. In addition, manufacturing time of the stent delivery system 200 can be shortened in some cases. Note that it is preferable that the stent 2 and the outer tube 6 do not overlap in the radial direction when the second start diameter is equal to or smaller than the diameter D of the proximal-side joint portion 16.

After the second diameter reduction step (after the second release step), the diameters (diameters after the recoil) of the stent 2 and the balloon 1 are smaller than the second start diameter of the second diameter reduction step. Therefore, when the compression is released in the second diameter reduction step, that is, in the second release step, a diameter (hereinafter, referred to as a second release diameter) of the hole portion 90 at the end of the second release step may be equal to or smaller than the diameter (hereinafter, referred to as the second start diameter) of the hole portion 90 at the start of the second diameter reduction step.

In the second release step of the second diameter reduction step performed last, the opening diameter of the crimping head 9 may be enlarged to be equal to or larger than the start diameter in order to easily take out (remove) the stent 2 and the balloon 1 from the crimping head 9.

As described above, the second diameter reduction step is preferably repeated twice (two times) or more, but the repetition of the second diameter reduction step may be ended in the following cases.

As an example, the repetition of the second diameter reduction step is preferably ended when the diameter of the stent 2 after the recoil occurring after the second diameter reduction step is equal to or smaller than a predetermined value (as an example, 1.02 mm). That is, the repetition of the second diameter reduction step may be ended if the diameter of the stent 2 is decreased to the predetermined value as a target value. As a result, the diameter can be efficiently decreased.

In addition, the repetition of the second diameter reduction step is preferably ended when a change rate (decrease rate) of the diameter of the stent 2 in a case where a load of 5 N or more and 10 N or less (5 N to 10 N) per length of 1 mm in the axial direction of the stent 2 is applied inward in the radial direction of the stent 2 becomes a predetermined amount x (%) or less. As a result, the diameter can be efficiently decreased.

Here, the change rate of the diameter of the stent 2 is a value obtained by dividing an absolute value of a difference Δd, obtained by subtracting a diameter d2 of the stent 2 after the application of the above load from a diameter d1 of the stent 2 before the application of the above load, by the diameter d1 and multiplying the division result by 100.

In a case where the second compression step in the second diameter reduction step is performed by applying a load of 5 N or more and 10 N or less (5 N to 10 N) per length of 1 mm in the axial direction of the stent 2 inward in the radial direction to the stent 2, the diameter d1 is a diameter of the stent 2 immediately before the start of the second diameter reduction step, and the diameter d2 is a diameter of the stent 2 after recoil occurring after the second diameter reduction step.

The predetermined amount x is, for example, 1.54% or less, preferably 1.44% or less, more preferably 0.66% or less, and still more preferably 0.60% or less. The predetermined amount x is allowed to be 0.08% or more.

In a case where the diameter reduction of the stent 2 in the second diameter reduction step is performed with a load of 5 N or more and 10 N or less (5 N to 10 N) per length of 1 mm in the axial direction of the stent 2, an amount of the decrease in the diameter of the stent 2 in this step corresponds to the difference Δd, and the diameter d2 is a diameter of the stent 2 immediately after the step.

In the crimping method according to the present embodiment, in the process of crimping the stent 2 to the balloon 1, a pressurization step of supplying fluid to the balloon 1 to inflate the balloon 1 and pressing the outer peripheral surface of the balloon 1 against the inner side of the stent 2 may be performed. Through the pressurization step, the amount of the balloon 1 pinched by the stents 2 increases, and the retention is improved. In addition, adhesion between the surface of the stent 2 on the inner side in the radial direction and the outer surface of the balloon 1 is improved, and the retention is improved.

The pressurization step may be performed before the first diameter reduction step, during the first diameter reduction step, or during the second diameter reduction step. As an example, the pressurization step may include a pre-pressurization step performed before the start of the first diameter reduction step (first compression step), an intermediate pressurization step performed during the first compression step, a post-pressurization step (first post-pressurization step) performed during the first maintenance step, and a post-pressurization step (second post-pressurization step) performed during the second maintenance step.

The pressurization step preferably includes the pre-pressurization step, the intermediate pressurization step, and the first post-pressurization step. As a result, the retention is improved. In this case, the pre-pressurization step, the intermediate pressurization step, and the first post-pressurization step are preferably a series of pressurization steps that are continued from before the start of the first compression step to the first maintenance step. As a result, the retention is further improved. Hereinafter, the series of pressurization steps continued from before the start of the first compression step to the first maintenance step is sometimes referred to as a first pressurization step.

In addition, the pressurization step preferably includes the second post-pressurization step. As a result, the retention is improved.

The pressurization step may include the first pressurization step and the second post-pressurization step, which may more favorably improve the retention.

When the pressurization step is performed, the second diameter reduction step is preferably repeated twice (two times) or more, which may improve the retention. When the first pressurization step or the second post-pressurization step is performed, it is preferable to further perform the second diameter reduction step in which no pressurization step is performed after the pressurization step. It is preferable that the second diameter reduction step including no pressurization step performed after the pressurization step is repeated two times or more, and preferably three to five times, which may further improve the retention.

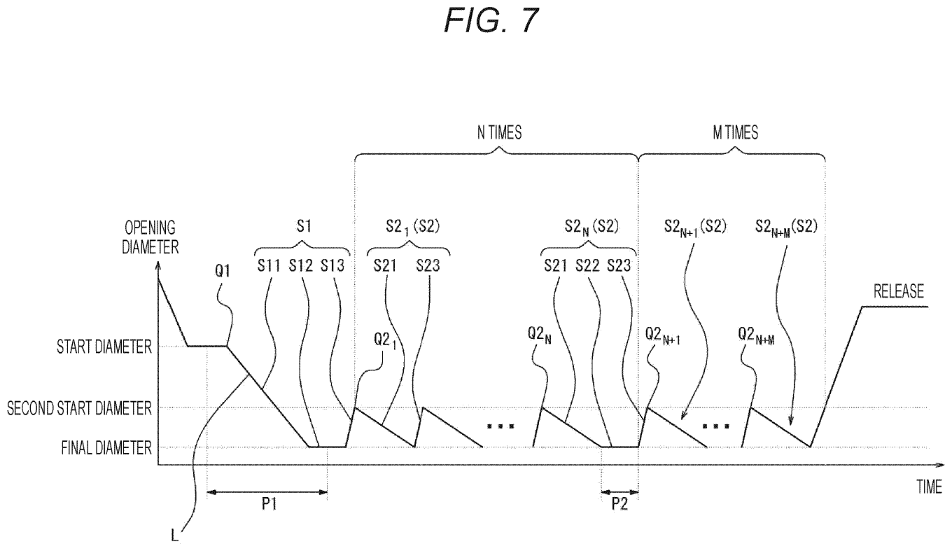

FIG. 7 illustrates a graph illustrating an example of the method (crimping method) for manufacturing the stent delivery system described above in the relationship between the opening diameter of the crimping head 9 (see FIGS. 5 and 6 above) of the crimping device and step elapsed time. In the graph of FIG. 7, the vertical axis represents the opening diameter of the crimping head 9 (opening diameter in FIG. 7), and the horizontal axis represents the step elapsed time in the method for manufacturing the stent delivery system. In the graph of FIG. 7, a solid line L indicates the opening diameter of the crimping head 9.

In FIG. 7, reference sign S1 denotes the first diameter reduction step. Reference sign S11 denotes the first compression step. Reference sign S12 denotes the first maintenance step. Reference sign S13 denotes the first release step. Reference sign Q1 denotes the start of the first diameter reduction step.

In FIG. 7, reference sign S2 denotes the second diameter reduction step. In the example illustrated in FIG. 7, the second diameter reduction step is performed N+M times (where N and M are natural numbers). Reference sign S2; such as reference sign S21 indicates the i-th (where i is a natural number less than or equal to N+M) second diameter reduction step. Reference sign S21 denotes the second compression step. Reference sign S22 denotes the second maintenance step. Reference sign S23 denotes the second release step. In addition, reference sign Q2i indicates the start of the i-th second diameter reduction step.

The second start diameter in the second diameter reduction step performed first (for the first time) is the same as the first release diameter in the first diameter reduction step. In the example illustrated in FIG. 7, the final diameter (second diameter) in the second diameter reduction step is the same as the final diameter (first diameter) in the first diameter reduction step. In the example illustrated in FIG. 7, the second release diameter in the second diameter reduction step excluding the last second diameter reduction step is the same as the second start diameter in the second diameter reduction step. The second release diameter in the last second diameter reduction step is set to be equal to or larger than the first start diameter, and crimping is terminated.

In FIG. 7, reference sign P1 denotes the first pressurization step. Reference sign P2 denotes the second post-pressurization step. In the example illustrated in FIG. 7, the first pressurization step includes the pre-pressurization step, the intermediate pressurization step, and the first post-pressurization step. The first pressurization step is a series of pressurization steps that are continued from before the start of the first compression step to the first maintenance step.

In the example illustrated in FIG. 7, after the second diameter reduction step (S2_{N}) in which the second post-pressurization step is performed, the second diameter reduction step (S2_{N+1} to S2_{N+M}) in which the second post-pressurization step is not performed is performed M times.

### EXAMPLES

Hereinafter, a stent delivery system and a method for manufacturing the same will be described based on Examples.

### Example 1

In this Example, according to the crimping method described above, in a state where a balloon disposed on a shaft having an inner tube and an outer tube was inserted into a tube of a stent having a diameter (outer diameter) of 2.0 mm, the stent and the balloon were inserted into a crimping head of a crimping device and crimped to crimp the stent on the balloon. Then, a diameter of the crimped stent was measured. As the balloon and the stent, “Ultimaster Nagomi®” manufactured by Terumo Corporation was used.

The balloon used in this Example is fused and joined to an outer peripheral surface of the outer tube on the proximal side, and a diameter (outer diameter) of such a proximal-side joint portion is 1.0 mm. The balloon is fused and joined to an outer peripheral surface of the inner tube on the distal side, and a diameter (outer diameter) of such a distal-side joint portion is 0.6 mm. The balloon was folded in advance to have three wings prior to insertion into the stent. The balloon is made of a polyamide resin. Note that the inner tube has an outer diameter of 0.25 mm, and the outer tube has an outer diameter of 0.65 mm.

The stent used in this Example is a wire mesh stent made of L-605 alloy.

Prior to the start of a first diameter reduction step, first, the stent was temporarily fixed to the balloon. This temporary fixing was performed by inserting the stent and the balloon into the crimping head of the crimping device and lightly crimping the stent and the balloon in a state where the balloon disposed on the shaft was inserted into the tube of the stent.

A start diameter in the first diameter reduction step is the same as an outer diameter of the stent after the above temporary fixing and is smaller than a diameter (2 mm) of the stent before the above temporary fixing.

A final diameter in a first compression step was set to 0.48 mm, which is smaller than the diameter of the proximal-side joint portion, to be smaller than the diameter of the proximal-side joint portion.

The first compression step was performed until reaching the final diameter while maintaining a state where a load of 5 N or more and 10 N or less (5 N to 10 N) per length of 1 mm in the axial direction of the stent was applied inward in the radial direction of the stent by sequentially reducing a hole diameter of the crimping head. The first compression step required approximately 30 seconds. In this Example, a first maintenance step was omitted. After the first compression step, a first release step was performed. A first release diameter was 1.2 mm larger than the diameter of the proximal-side joint portion.

After the first diameter reduction step, a second diameter reduction step was repeated 15 times. Second start diameters in all the second diameter reduction steps were 1.2 mm which is the same as the first release diameter. That is, second release diameters in the first to fourteenth second diameter reduction steps were 1.2 mm which is the same as the first release diameter. A second release diameter in the fifteenth second diameter reduction step was made larger than the start diameter. In this Example, a second maintenance step was omitted in all the second diameter reduction steps.

Similarly to the first compression step, a second compression step in the second diameter reduction step was performed until reaching the final diameter while maintaining a state where a load of 5 N or more and 10 N or less (5 N to 10 N) per length of 1 mm in the axial direction of the stent was applied inward in the radial direction of the stent by sequentially reducing a hole diameter of the crimping head. Each of the second compression steps required approximately 20 seconds.

After the first diameter reduction step and after each of the second diameter reduction steps, the diameter of the stent crimped on the balloon was measured. Measurement results are shown in Table 1. Note that “Crimping time” in Table 1 is the number of times counted with the first diameter reduction step as the first time and the second diameter reduction steps and the subsequent steps as the second and subsequent times. That is, in Table 1, compression and release in each of the first diameter reduction step and the second diameter reduction step are defined as one cycle of a crimping operation, and a cumulative number of the crimping operations is shown as the crimping time. That is, a diameter of the first crimping operation in Table 1 is a diameter of the stent immediately after the first diameter reduction step. In addition, a diameter of the second crimping operation in Table 1 is a diameter of the stent immediately after the first second diameter reduction step, and the same applies to the third and subsequent crimping operations.

TABLE 1

| Crimping time (time) | Diameter | Difference | Absolute value (%) |
|---|---|---|---|
| 1 | 1.075 | — | — |
| 2 | 1.050 | 2.273 | 2.273 |
| 3 | 1.035 | 1.444 | 1.444 |
| 4 | 1.028 | 0.650 | 0.650 |
| 5 | 1.028 | 0.010 | 0.010 |
| 6 | 1.023 | 0.557 | 0.557 |
| 7 | 1.022 | 0.044 | 0.044 |
| 8 | 1.018 | 0.397 | 0.397 |
| 9 | 1.014 | 0.403 | 0.403 |
| 10 | 1.010 | 0.414 | 0.414 |
| 11 | 1.013 | −0.281 | 0.281 |
| 12 | 1.008 | 0.469 | 0.469 |
| 13 | 1.013 | −0.459 | 0.459 |
| 14 | 1.013 | −0.090 | 0.090 |
| 15 | 1.007 | 0.588 | 0.588 |
| 16 | 1.004 | 0.332 | 0.332 |

Table 1 shows a change rate (%) before and after the crimping operation together. This change rate is a value obtained by dividing an absolute value (Absolute value in Table 1) of a difference (Difference in Table 1) obtained by subtracting a diameter of the stent after each crimping operation described above from a diameter of the stent immediately before the crimping operation by the diameter of the stent immediately before the crimping operation and multiplying the division result by 100. For example, the change rate when the number of crimping times is three is a value obtained by dividing an absolute value of a difference obtained by subtracting a diameter of the stent after the third crimping operation from a diameter of the stent immediately before the third crimping operation, that is, after the second crimping operation by a diameter of the stent after the second crimping operation, and multiplying the division result by 100.

As shown in Table 1, the change rate decreased to about 1.44% after the third crimping time, and the stent was decreased in diameter. Therefore, it is considered that the change rate needs to be at least 1.54% or less to decrease the diameter of the stent. It is considered that the change rate is preferably 1.44% or less for an appropriate decrease in the diameter of the stent. When the diameter of the stent is sufficiently decreased, the balloon is sufficiently pinched by the stent 2, so that it can be determined that retention of the balloon has reached a practically necessary level.

In this Example, the final diameter in the first compression step is 0.48 mm, which is smaller than the diameter of the proximal-side joint portion, to be smaller than the diameter of the proximal-side joint portion, but it is considered that the setting of the final diameter in the first compression step in this manner also contributes to the decrease in the diameter of the stent after the third crimping time.

In addition, in this Example, the final diameter in the second compression step is 0.48 mm, which is smaller than the diameter of the proximal-side joint portion, to be smaller than the diameter of the proximal-side joint portion, but it is considered that the setting of the final diameter in the second compression step in this manner also contributes to the decrease in the diameter of the stent after the third crimping time.

After the fourth crimping time, the change rate reaches about 0.66% or less, and the stent is sufficiently decreased in diameter. Therefore, it is considered that the stent can be sufficiently decreased in diameter when the change rate is 0.66% or less.

After the fifth crimping time, the change rate reaches about 0.60% or less, and the stent is further sufficiently decreased in diameter. Therefore, it is considered that the stent can be further sufficiently decreased in diameter when the change rate is 0.60% or less. Note that the change rate is 0.08% or more in the ninth and subsequent crimping times.

Based on the change rate after the fifth crimping time, it is considered that the stent can be sufficiently decreased in diameter even if the repetition of the second diameter reduction step is ended at the time when the change rate reaches 0.60% or less.

Focusing on the number of crimping times, it is considered that a sufficient decrease in diameter is achieved if the second diameter reduction step is performed four times or more, and preferably eight times or more, and it is sufficient if the second diameter reduction step is performed nine times or more since the change rate is approximately constant in the fifth and subsequent crimping times, more specifically, in the ninth and subsequent crimping times.

For example, if the second diameter reduction step is performed four times or more and the change rate is controlled to 0.60% or less, it is considered that a reliable decrease in diameter can be achieved.

The finally obtained diameter of the stent was smaller than the diameter of the proximal-side joint portion and larger than the diameter of the inner tube.

Note that no pinhole was observed in the balloon in the last second diameter reduction step.

### Example 2

In Example 2, the stent was crimped on the balloon by performing processing up to the fifteenth second diameter reduction step in the same manner as in Example 1 except that lots of the stent and the balloon were changed. In Example 2, a diameter of the stent and retention (a holding force) after the fifteenth second diameter reduction step were measured. In Example 2, six crimped stents were manufactured by the same manufacturing method, and an average value of diameters of five stents and an average value of the retention (holding force) of four stents among the six stents were acquired. In addition, variations in diameter and variations in retention of the crimped stents were evaluated based on standard deviations. Table 2 shows the average value of diameters (mm), the standard deviation of diameters (mm), the average value of retention (N), and the standard deviation of retention (N) of the crimped stents.

TABLE 2

| | Average value | Standard deviation | Average value | Standard deviation |
|---|---|---|---|---|
| | of diameters (mm) | of diameters (mm) | of retention (N) | of retention (N) |
| Example 2 | 0.897 | 0.007 | 1.22 | 0.29 |
| Example 3 | 0.886 | 0.009 | 1.38 | 0.22 |
| Example 4 | 0.882 | 0.007 | 1.53 | 0.23 |

Note that the retention is the force of holding the stent in a state of being crimped on the balloon. The retention in this Example is a value measured in accordance with ASTM F2394-07 (initial peak displacement force described in Reapproved 2022), and is a pulling force when the stent falls off the balloon when the stent is pulled in the axial direction with respect to the balloon.

### Example 3

In Example 3, an average value of diameters of the stent and an average value of the retention (holding force) were acquired in the same manner as in Example 2 except that the second maintenance step was performed only in the fifteenth second diameter reduction step, and a pressurization step of supplying fluid to the balloon to inflate the balloon and pressing the balloon against the inside of the stent was performed during the second maintenance step, and variations thereof were evaluated. Table 2 also shows these results.

In this Example, the second maintenance step in the fifteenth second diameter reduction step was performed for 30 seconds. That is, in the fifteenth second diameter reduction step, a hole diameter of the crimping device was maintained at a final diameter for 30 seconds, as the second maintenance step, after a compression step. After the second maintenance step, a second release step was performed to make the hole diameter of the crimping device larger than a start diameter.

The pressurization step was started at the same time as the start of the second maintenance step and ended at the same time as the end of the second maintenance step. That is, the pressurization step was performed for 30 seconds. In the pressurization step, the inside of the balloon was pressurized and maintained at 1.4 MPa, and the pressure in the balloon was released at the end of the pressurization step.

### Example 4

In Example 4, processing until the second maintenance step in the fifteenth second diameter reduction step and the pressurization step performed simultaneously with the second maintenance step was performed in the same manner as in Example 3. Thereafter, a difference from Example 3 is that the second diameter reduction step was further repeated four times after the second maintenance step in the fifteenth second diameter reduction step and the pressurization step performed simultaneously with the second maintenance step were performed. The last (nineteenth) second diameter reduction step was performed in the same manner as the last (fifteenth) second diameter reduction step in Example 2. For the evaluation of the stent crimped on the balloon, an average value of diameters of the stent and an average value of the retention (holding force) were acquired in the same manner as in Examples 2 and 3, and these variations were evaluated. Table 2 also shows these results.

As shown in Table 2, in the comparison among Examples 2 to 4, a decrease in diameter of the stent is achieved regardless of the presence or absence of the pressurization step. However, in Examples 3 and 4 in which the pressurization step was performed, values of the retention are higher than that in Example 2 in which the pressurization step was not performed, and it can be seen that the effect of improving the retention is obtained by the pressurization step. In addition, in Examples 3 and 4 in which the pressurization step was performed, the variations in retention were also reduced as compared with Example 2 in which the pressurization step was not performed, and it can be seen that the effect of stabilizing the retention is obtained by the pressurization step.

From these results, it is considered that an amount of the balloon pinched by the stent was increased by the pressurization step, and the retention was improved. In addition, it is considered that adhesion between a surface of the stent on the inner side in the radial direction and an outer surface of the balloon was improved by the pressurization step, and the retention was improved.

In addition, considering the presence or absence of the second diameter reduction step after the pressurization step, the value of the retention is higher in Example 4 in which the second diameter reduction step after the pressurization step was performed than in Example 3 in which the second diameter reduction step after the pressurization step was not performed, and it can be seen that the effect of improving the retention is obtained by performing the second diameter reduction step in which the pressurization step was not performed after the second diameter reduction step in which the pressurization step was performed.

From these results, it is considered that the amount of the balloon pinched by the stent was further increased by the second diameter reduction step after the pressurization step, and the retention was improved. In addition, it is considered that the adhesion between the surface of the stent on the inner side in the radial direction and the outer surface of the balloon was further improved by the second diameter reduction step after the pressurization step, and the retention was improved.

As described above, it is possible to provide the stent delivery system and the method for manufacturing the same that achieve a decrease in diameter of a balloon catheter on which the stent is crimped.

### Another Embodiment

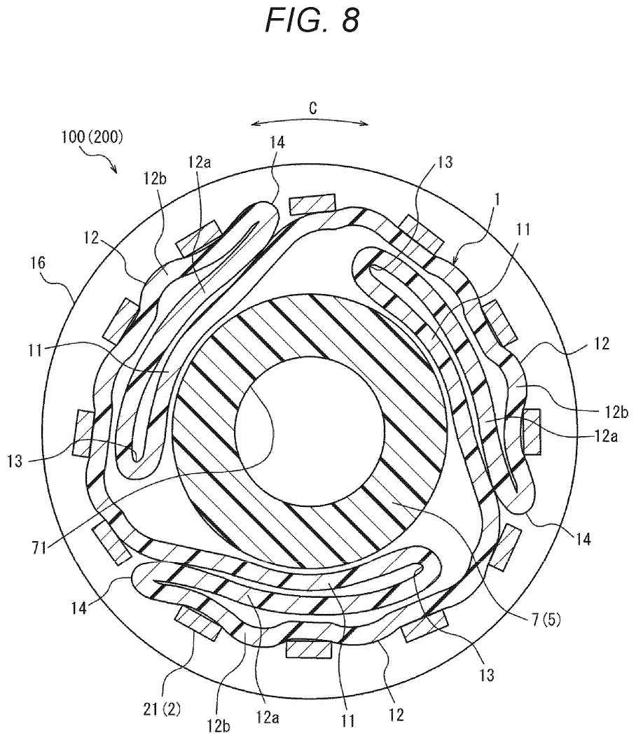

(1) In the above embodiment, the stent delivery system 200 (see FIG. 1 and the like) in which the diameter of the stent 2 is larger than the diameter D of the proximal-side joint portion 16 in the folded state of the balloon 1 (before the expansion of the stent 2) has been described as an example. In the stent delivery system 200, however, the diameter of the stent 2 may be equal to or smaller than the diameter D of the proximal-side joint portion 16 in the folded state of the balloon 1 as illustrated in FIG. 8. Note that FIG. 8 illustrates a cross section similar to FIG. 2. In this case, the diameter of the stent 2 is made equal to or larger than the diameter of the inner tube 7.

Note that the embodiments disclosed in the present specification are examples, and the embodiments of the present disclosure are not limited thereto, and can be appropriately modified within a scope not departing from the object of the present disclosure.

The detailed description above describes a method for manufacturing a stent delivery system and a stent delivery system. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A method for manufacturing a stent delivery system including a balloon catheter that includes a balloon inflated or deflated by supply or discharge of fluid and a shaft that supports the balloon and supplies or discharges the fluid, and a stent disposed on the balloon and formed in a tubular shape, the balloon being joined to the shaft at an end in an extending direction of the shaft, the method comprising:
a first diameter reduction step of compressing the stent inward in a radial direction of the stent in a state where the balloon is inserted into a tube of the stent to reduce diameters of the stent and the balloon, and then releasing the compression,
wherein the stent is compressed to a first diameter in the first diameter reduction step, and
the first diameter is equal to or smaller than a diameter of a joint portion where a proximal portion of the balloon is joined to the shaft.

2. The method for manufacturing a stent delivery system according to claim 1, further comprising:
a second diameter reduction step performed after the first diameter reduction step, the second diameter reduction step of compressing the stent inward in the radial direction of the stent to reduce the diameters of the stent and the balloon, and then releasing the compression,
wherein the stent is compressed to a second diameter in the second diameter reduction step, and
the second diameter is equal to or smaller than the diameter of the joint portion wherein the proximal portion of the balloon is joined to the shaft.

3. The method for manufacturing a stent delivery system according to claim 2, wherein the second diameter reduction step is repeated two or more times.

4. The method for manufacturing a stent delivery system according to claim 2, wherein a start diameter at the compression in the second diameter reduction step is equal to or smaller than the diameter of the joint portion wherein proximal portion of the balloon is joined to the shaft.

5. The method for manufacturing a stent delivery system according to claim 4, wherein when a change rate of the diameter of the stent when a load of 5 N or more and 10 N or less per length of 1 mm in an axial direction of the stent is applied inward in the radial direction of the stent is 0.60% or less, repetition of the second diameter reduction step is ended.

6. A stent delivery system comprising:
a balloon catheter that includes a balloon inflated or deflated by supply or discharge of fluid and a shaft that supports the balloon and supplies or discharges the fluid; and
a stent disposed on the balloon and formed in a tubular shape,
wherein an end of the balloon in an extending direction of the shaft is joined to the shaft, and
a diameter of the stent is equal to or smaller than a diameter of a joint portion where a proximal portion of the balloon is joined to the shaft.

7. The stent delivery system according to claim 6, wherein a diameter of the stent where a distal portion of the balloon is joined to the shaft is 0.3 mm to 1.5 mm and a length in an axial direction of 0.5 mm to 5 mm.

8. The stent delivery system according to claim 6, wherein the diameter of the stent where the proximal portion of the balloon is joined to the shaft is 0.5 mm to 1.8 mm and a length in an axial of 1 mm to 6 mm.

9. The stent delivery system according to claim 6, wherein the stent and an outer tube of the balloon do not overlap in a radial direction when the diameter of the stent is equal to or smaller than the diameter of joint portion where proximal portion of the balloon is joined to the shaft.

10. A method for manufacturing a stent delivery system including a balloon catheter that includes a balloon inflated or deflated by supply or discharge of fluid and a shaft that supports the balloon and supplies or discharges the fluid, and a stent disposed on the balloon and formed in a tubular shape, the balloon being joined to the shaft at an end in an extending direction of the shaft, the method comprising:
compressing the stent inward a first time in a radial direction of the stent in a state where the balloon is inserted into a tube of the stent to reduce diameters of the stent and the balloon, and then releasing the compression.

11. The method for manufacturing the stent delivery system according to claim 10, wherein the stent is compressed to a first diameter during the compression of the stent inward the first time, and the first diameter is equal to or smaller than a diameter of a joint portion where a proximal portion of the balloon is joined to the shaft.

12. The method for manufacturing the stent delivery system according to claim 10, further comprising:
compressing the stent inward a second time in the radial direction of the stent to reduce the diameters of the stent and the balloon, and then releasing the compression.

13. The method for manufacturing the stent delivery system according to claim 12, wherein the stent is compressed to a second diameter during the compression of the stent inward the second time, and the second diameter is equal to or smaller than the diameter of the joint portion where the proximal portion of the balloon is joined to the shaft.

14. The method for manufacturing the stent delivery system according to claim 13, further comprising:
repeating the compression of the stent inward the second time in the radial direction of the stent to reduce the diameters of the stent and the balloon, and then releasing the compression at least twice.

15. The method for manufacturing the stent delivery system according to claim 13, further comprising:
repeating the compression of the stent inward the second time in the radial direction of the stent to reduce the diameters of the stent and the balloon, and then releasing the compression between 3 times to 20 times.

16. The method for manufacturing the stent delivery system according to claim 13, wherein a start diameter at the compression of the stent inward the second time is equal to or smaller than the diameter of the joint portion where the proximal portion of the balloon is joined to the shaft.

17. The method for manufacturing the stent delivery system according to claim 16, wherein when a change rate of the diameter of the stent when a load of 5 N or more and 10 N or less per length of 1 mm in an axial direction of the stent is applied inward in the radial direction of the stent is 0.60% or less, repetition of the compression of the stent inward the second time is ended.

18. The method for manufacturing the stent delivery system according to claim 17, wherein a diameter of the stent is equal to or smaller than a diameter of a joint portion where a proximal portion of the balloon is joined to the shaft after the ending of the repetition of the compression of the stent inward the second time.

19. The method for manufacturing the stent delivery system according to claim 10, further comprising:
preforming the compression of the stent inward the first time in the radial direction with a load of 5 N or more and 10 N or less per length of 1 mm in an axial direction of the stent.

20. The method for manufacturing the stent delivery system according to claim 12, further comprising:
preforming the compression of the stent inward the second time in the radial direction with a load of 5 N or more and 10 N or less per length of 1 mm in an axial direction of the stent.
